(19) United States
(12) Patent Application Publication
(10) Pub. No.: US 2010/0262219 A1
(43) Pub. Date: Oct. 14, 2010

(54) DEVICE FOR ANGIOPLASTY WITH AN EMBOLIZATION PROTECTION COMPONENT AND METHOD THEREFOR

(76) Inventor: Aharon FRIMERMAN, Yavne (IL)

Correspondence Address: EDWARD LANGER;c/o SHIBOLETH YISRAELI ROBERTS ZISMAN & CO., 1 PENN PLAZA-SUITE 2527, NEW YORK, NY, 10119, US

(21) Appl. No.: 12/758,850

(22) Filed: Apr. 13, 2010

Related U.S. Application Data
(60) Provisional application No. 61/212,599, filed on Apr. 14, 2009.

(51) Int. Cl. A61F 2/06; A61B 17/22; A61M 29/02

(52) U.S. Cl. 623/1.11; 606/200; 606/194

(57) ABSTRACT

A device and method for balloon and stent angioplasty for treating atherosclerosis with a distal embolization protection component. The device includes an endoluminal dilatation balloon formed on a shaft having at least one lumen, a flexible filter mounted on the shaft, distal to the balloon and a filter deployment memory ring circling the proximal opening of the flexible filter for the purpose of controlling deployment and collapsing of the filter. When the endoluminal dilatation balloon is inflated for the purpose of dilating plaque that is blocking a blood vessel, the flexible filter deployed prior to balloon inflation, traps atherosclerotic debris and thrombi emboli that are released from the plaque, thus allowing blood flow to continue without interference during the angioplasty procedure. At the end of the procedure the flexible filter, containing the debris, is pulled and collapsed into a small profile and is pulled out of the artery as an integrated unit with the balloon. The device may or may not have a stent pre-crimped over the dilatation balloon.

### FIELD OF THE INVENTION

The present invention relates generally to balloon angioplasty, and more particularly to angioplasty devices providing protection against embolization.

### BACKGROUND OF THE INVENTION

Coronary artery disease (CAD) affects almost 1.3 million Americans per year, making it the most common form of heart disease. CAD most often results from a condition known as atherosclerosis, which is the most common form of arteriosclerosis—hardening of the arteries.

Atherosclerosis occurs when plaque forms inside the arteries. Plaque is made of cholesterol, fatty compounds, calcium and fibrin. As the plaque builds up, the artery narrows, making it more difficult for blood to flow through the arteries. When atherosclerosis occurs in a coronary artery, blood flow to the heart muscle is impeded. In time, the narrowed or blocked artery can lead to angina pectoris, Myocardial Infarct (MI) and possibly death. A similar processes can affect other arteries, such as thecarotid, intra-cerebral, and renal arteries, as well as arteries of the upper and lower limbs, leading to stroke, renal failure, hypertension and malfunction of the limbs, depending on artery affected.

In order to reduce the risk of artery disease and its complications, one option is opening the artery narrowing induced by atherosclerosis, utilizing intra-lumen balloon inflation and/or intra-lumen stent deployment. A balloon or stent angioplasty is performed as follows:

A guiding catheter is inserted through a small skin incision into an artery and advanced to the origin of the coronary artery. Next, a guide wire is advanced through the guiding catheter into the coronary artery and across the blockage site. Then, a long, thin catheter that has a small balloon on its tip (balloon catheter) is pushed over the guide wire. The balloon is inflated at the blockage site in the artery to flatten or compress the plaque against the artery wall, and it is then deflated and withdrawn. Thus, the blockage is removed from at least a portion of the arterial lumen, and blood can flow more freely through the artery.

Another method for opening plaque blockage is stent deployment. A stent is a small mesh-like tube made of metal. The stent is pre-crimped over a balloon on the tip of a balloon catheter and pushed over the guide wire (as explained above) to the blockage site, where it is then expanded and deployed by the pressure of the balloon inflation at the narrowed site, and as so, acts as a support or scaffold, keeping the vessel open. Stent procedures are usually used along with balloon angioplasty. The first balloon inflation prepares the narrowed site, and enables stent insertion and deployment by another balloon on which the stent is crimped on.

Such procedures may be used to open narrowing in arteries including coronary, carotid, intra-cranial, and renal arteries, and peripheral arteries in the legs and arms.

Unfortunately, these procedures pose a great risk to the patient as emboli, generated by the balloon inflation or stent deployment that crush the frail atherosclerotic plaque, or thrombotic material, may be released during them. Once released, these emboli have a high likelihood of getting lodged into the smaller vessels at a point of constriction downstream of their release point, causing the vessels to become occluded, preventing blood flow.

These adverse events may cause severe damage to the treated organ like myocardial infarction, stroke, renal failure or limb malfunction.

There are several known embolization protection devices based mainly on an additional small proximal or distal occluding balloon with some debris removal system, or a small filter attached to the guide wire with deployment and retrieval systems.

Examples of known embolization protection devices, include:

U.S. patent application Ser. Nos. 10/348,137, 11/387,366, 11/566,473 to Wholey et al;

U.S. patent application Ser. Nos. 11/763,118, 10/997,803 to Sachar et al;

U.S. patent application Ser. No. 11/271,653 to Blix et al;

U.S. patent application Ser. Nos. 09/952,375 to Fischell et al; and

U.S. patent application Ser. Nos. 09/845,162 to Wahr.

The above-mentioned devices have many limitations. The occluding balloon-based type induces ischemia since the occluding balloon stops the distal blood flow for a relatively long period. Moreover, the need for a special debris extraction catheter prolongs the procedure and increases its complexity.

The over-the-wire filter devices have a special guide wire with inferior crossing ability compared to the regular wires. The extra delivery sheath covers the filter prior to its deployment, increasing the wire profile, reducing its crossing ability and because of its larger size, may cause embolization. There is also difficulty delivering the filter to a proper site distally to the lesion and difficulty in maintaining its position during the procedure. The need for another retrieval system prolongs the whole procedure time.

It is no wonder that several clinical randomized studies showed that in a group of patients in which these devices were used, the overall adverse events were significantly higher than in the control group where such devices were not applied.

Therefore, there is a need for a new embolization protection device which will decrease the risks of distal embolization during use of intra-lumen devices.

### SUMMARY OF THE INVENTION

Accordingly, it is a principal object of the present invention to overcome the limitations of prior art embolization protection devices by providing a distal embolization protection component as an integral part of the angioplasty device, and a simple deployment/retrieval method that does not change or interfere with the current angioplasty technique, while trapping any emboli that are released, and allowing blood to continue flowing without interference during the angioplasty procedure.

In accordance with a preferred embodiment of the present invention, there is provided an angioplasty device for providing protection against distal embolization of atherosclerotic debris and thrombi emboli resulting from an angioplasty procedure, said device comprising an endoluminal dilatation balloon mounted on a shaft, said shaft having at least one lumen; a flexible filter mounted on said shaft, distal to said balloon; and a filter deployment ring encircling a proximal end of said flexible filter for the purpose of controlling deployment and collapsing of said filter, said filter deployment ring comprising shape memory metal, wherein said flexible filter, when deployed, traps said atherosclerotic debris and thrombi emboli thus allowing blood flow to continue without interference during said angioplasty procedure.

The inventive embolization protection device comprises a shaft having a dilatation balloon (with or without a stent) mounted on it, and a flexible filter mounted on the shaft distally to the balloon, and a filter deployment ring disposed around the proximal opening of the flexible filter. The purpose of the filter deployment ring is to open the proximal end of the filter at the beginning of the procedure. This will deploy the filter opening to the full size of the artery circumference at the treatment site to seal the artery so that the atherosclerotic debris and thrombi emboli released from the plaque during the procedure, will be trapped inside the filter. The filter deployment ring will be used also for collapsing the filter at the end of the procedure, to a small profile, with the trapped emboli inside, for enabling retraction of the balloon catheter with the attached filter as one unit.

In one embodiment, the endoluminal dilatation balloon may have a stent crimped over it.

The present invention is designed for equipping any intra-luminal balloon-based procedure (such as balloon angioplasty or stent deployment) with an integrated embolization protection component maintaining the current state of the art technique including the wiring system and balloon inflation-deflation method, without extra manipulation, hardware or overall procedure time. The new balloon-filter assembly is designed to replace all existing dilatation intra-luminal balloons as well as balloons used for stent deployment.

While the balloon, or balloon and stent are compressing and crushing the plaque constricting the blood flow, pieces of the plaque are released into the bloodstream and can become lodged in smaller vessels downstream to the angioplasty site, and block them. To avoid such a scenario, a filter is connected to the balloon catheter shaft, on its distal side, and traps any emboli that are released, while allowing blood to continue flowing without interference.

In accordance with some embodiments of the present invention, the filter deployment ring is connected to a thin retraction filament extending through a lumen in the shaft and emerging from an opening in the shaft, distal to the balloon to avoid its entrapment by the stent.

The retraction filament may optionally extend through a lumen which is also an inflation fluid lumen. Alternatively, the retraction filament may extend through a dedicated lumen within the shaft.

In accordance with a second embodiment of the present invention, the retraction filament that is attached to the filter deployment ring, emerges from an opening in the shaft, proximal to the balloon, and thereby the retraction filament runs over the entire length of the balloon. The device for a blood vessel angioplasty balloon, according to this embodiment, cannot have a stent pre-crimped over it because the retraction filament and filter can get tangled with a stent, if it were there, at the stage of the device withdrawal.

According to some embodiments of the present invention, the filter deployment ring is tightened by pulling the retraction filament connected to the filter deployment ring. The tightened filter deployment ring causes the filter to close, containing in it trapped atherosclerotic debris and thrombi emboli.

According to some embodiments, the device of the present invention further comprises a separate retraction filament tube proximal to the balloon, and joining the hollow shaft, wherein the retraction filament extends through the shaft, and a locking device enabling and disabling forward and backward movement of the retraction filament, wherein the locking device locks the retraction filament onto the retraction filament tube through which the retraction filament passes.

According to some embodiments, the device further comprises a balloon inflation/deflation device configured as a syringe having a plunger proximal to the shaft, and connected to the shaft via an inflation tube, wherein the balloon inflation/deflation device injects inflation fluid through the inflation tube into the balloon for inflating it, and withdrawing fluid from the balloon for deflating it.

Optionally, the balloon is reversibly and repeatedly inflatable.

According to some embodiments, the filter deployment ring is deployed by unlocking the locking device and thereby releasing the thin retraction filament connected to the filter deployment ring, such that the ring returns to its original shape by memory.

According to some embodiments, the device of the present invention further comprises a safety stopper for ensuring that deployment of the filter occurs before balloon inflation, wherein the safety stopper is attached to the retraction filament and is situated at the connection point of the inflation tube with the shaft, thus blocking the entrance of inflation fluid for inflating the balloon, when the retraction filament is pulled and the filter is not yet deployed.

According to some embodiments, the safety stopper is pushed forward with the forward movement of the retraction filament when deploying the filter, and the stopper is then removed from the connection point of the inflation tube to said shaft so that said inflation fluid is allowed to flow through the shaft and inflate the balloon.

According to some embodiments, the shaft is wider in diameter beyond the connection point of the inflation tube to the shaft, for the purpose of accommodating the safety stopper.

According to some embodiments, the device further comprises a bulbous area formed in the shaft situated distally to the wider section of the shaft, wherein said bulbous area is large enough to contain the safety stopper once the filter is deployed and allow fluid to flow through it, and wherein distally to the bulbous area the shaft continues at a regular width.

The safety stopper optionally comprises at least one of silicon and rubber.

According to some embodiments, the length of the wider section of the shaft together with the length of the bulbous area is equal to the distance the retraction filament is pushed forward when the filter is deployed.

In some embodiments, the balloon is partially surrounded by the filter so that when the balloon is inflated it will further deploy the filter by pushing against it.

In some embodiments, the device further comprises a stent that is pre-crimped over the balloon, and deployment of the stent is achieved by inflation of the balloon.

In some embodiments, the diameter of the shaft is approximately two millimeters.

In some embodiments, the diameter of the balloon before inflation is 0.5 to 0.7 millimeters for coronary balloons, and the diameter of the balloon when inflated is approximately 2 to 4 millimeters, and the diameter of the balloon after deflation is approximately 2.5 millimeters.

In some embodiments, the diameter of the balloon for coronary stents is 1.1 to 1.5 millimeters.

In some embodiments, the filter deployment ring comprises a shape memory metal and the flexible filter is made of one of a shape memory metal and a very thin polymeric material.

In some embodiments, a diameter of the flexible filter before deployment is 0.5 to 1 millimeter in diameter, and the maximal size of said filter at the ring site when it is deployed is 2.5 to 5 millimeters in diameter.

According to some embodiments, the filter deployment ring comprises a shape memory metal, such as Nitinol.

In accordance with the preferred embodiments of the present invention there is provided a method for performing an angioplasty procedure and providing protection against distal embolization resulting in the angioplasty procedure, the method comprising:

inserting an endoluminal dilatation balloon mounted on a guide wire inside a hollow shaft into a blood vessel obstructed by plaque, the shaft having a flexible filter as an integral part, mounted thereon, distal to the balloon;

deploying the flexible filter by releasing a filter deployment ring disposed around a proximal end of the flexible filter for the purpose of controlling deployment and collapsing of the filter, and

inflating the dilatation balloon so that it expands and crushes the plaque,

wherein the deployed flexible filter, traps atherosclerotic debris and thrombi emboli that are released from the crushed plaque, thus allowing blood flow to continue without interference during the angioplasty procedure, and

wherein the filter deployment ring is then pulled to collapse the filter trapping within it released thrombi emboli.

The method for distal embolization protection during angioplasty comprises first inserting the device into the blocked artery at the plaque site, then deploying the flexible filter by releasing the filter deployment ring, which is disposed around the proximal end of the filter to the size of the artery circumference at the treatment site, sealing it from distal debris embolization, and then inflating the dilatation balloon which crushes the plaque. The dilatation balloon is then deflated, and by so doing, emboli are released from the plaque and are captured by the deployed filter, while allowing blood to flow freely through it. The procedure ends by pulling the filter deployment memory ring which collapses the filter to a small profile with the emboli trapped in it, enabling withdrawal of the device out of the artery.

A feature of the present invention is a safety stopper, which is optionally substantially conical in shape, and is responsible for assuring that the balloon inflation will occur only once the filter is deployed, and not prior to deployment. The stopper does not allow inflation of the balloon if the filter is not deployed yet. This feature eliminates the possibility of human error, since an error in the order of the deployment of the balloon and filter would cause emboli to be released from the plaque into the blood stream without a filter to trap it.

Additional features and advantages will become apparent from the following drawings and description.

### BRIEF DESCRIPTION OF THE DRAWINGS

For a better understanding of the invention with regard to the embodiments thereof, reference is made to the accompanying drawings, in which like numerals designate corresponding elements or sections throughout, and in which:

FIG. 1 is a prior art embolization protection device;

FIG. 2A shows a perspective view of a dilatation balloon with a distal embolization device;

FIG. 2B shows a cross-section along section lines A-A of an embodiment of the device of FIG. 2A;

FIG. 2C shows a cross-section along section lines A-A of an alternative embodiment of the device of FIG. 2A;

FIG. 3A shows a balloon inflation/deflation device, a retraction filament and a retraction filament locking device;

FIG. 3B shows the device of FIG. 3A with a bulbous modification on a shaft for accommodating a safety stopper;

FIG. 3C shows a safety stopper blocking balloon inflation;

FIG. 3D shows the safety stopper allowing balloon inflation;

FIG. 4 shows a dilatation balloon with an embolization protection device lodged in a restricted blood vessel;

FIG. 5 shows the balloon of FIG. 4 with the embolization protection device deployed;

FIG. 6 shows the balloon of FIG. 4 inflated;

FIG. 7 shows the balloon of FIG. 4 deflated and emboli are captured in the protection device;

FIG. 8 shows the balloon of FIG. 4 with the protection device closed and containing released emboli, retracting from the artery;

FIG. 9 shows the unrestricted blood vessel after treatment;

FIG. 10 shows a dilation balloon and stent with a distal embolization device lodged in a restricted blood vessel;

FIG. 11 shows the balloon and stent of FIG. 10 with the embolization protection device deployed;

FIG. 12 shows the balloon and stent of FIG. 10 inflated and deployed, respectively;

FIG. 13 shows the balloon and stent of FIG. 10 deflated and emboli are captured in the protection device;

FIG. 14 shows the balloon and stent of FIG. 10 with the protection device closed and containing released emboli, retracting from the artery;

FIG. 15 shows the unrestricted blood vessel, with stent, after treatment;

FIG. 16 shows a perspective view of a dilatation balloon with a distal embolization device, which cannot be used with a stent;

FIG. 17 shows the dilatation balloon of FIG. 16, lodged in a constricted blood vessel;

FIG. 18 shows the dilatation balloon of FIG. 16, with the embolization protection device deployed;

FIG. 19 shows the dilatation balloon of FIG. 16, with the balloon inflated;

FIG. 20 shows the dilatation balloon of FIG. 16, with the balloon deflated and emboli are captured in the embolization protection device;

FIG. 21 shows the dilatation balloon of FIG. 16, with the embolization protection device closed; and

FIG. 22 shows the blood vessel after treatment.

### DETAILED DESCRIPTION OF THE PREFERRED EMBODIMENTS

It is a principal object of the present invention to provide health care to an atherosclerosis patient by providing an angioplasty device comprising an endoluminal dilatation balloon, with or without a stent, and a flexible filter as an integral part of the angioplasty device. The filter allows blood to flow through it but can also capture and remove from the blood vessel matter like thrombi or atherosclerotic debris, thus preventing distal embolization.

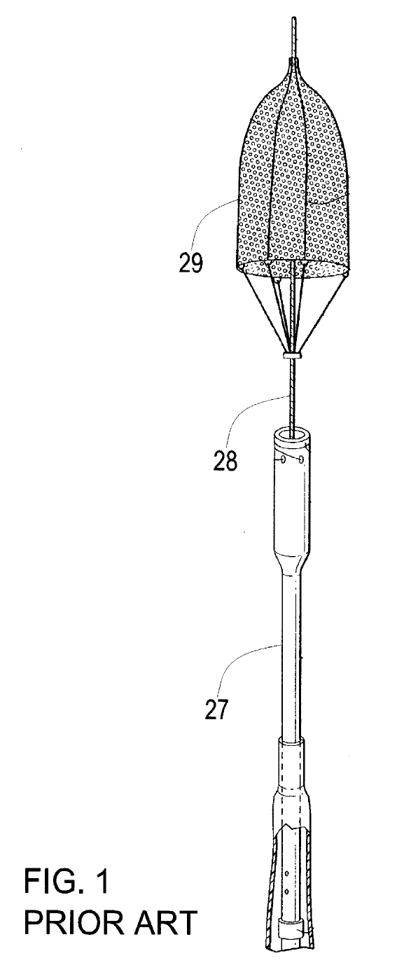

Referring now to FIG. 1, there is shown a prior art illustration of an embolization protection device. A filter assembly 29 is disposed on a guide wire 28, distal to a shaft 27. The filter 29 is not mounted directly on shaft 27, therefore deployment is more difficult, as explained in the background. This is in contrast to the inventive solution as disclosed in the description of the present invention hereinbelow.

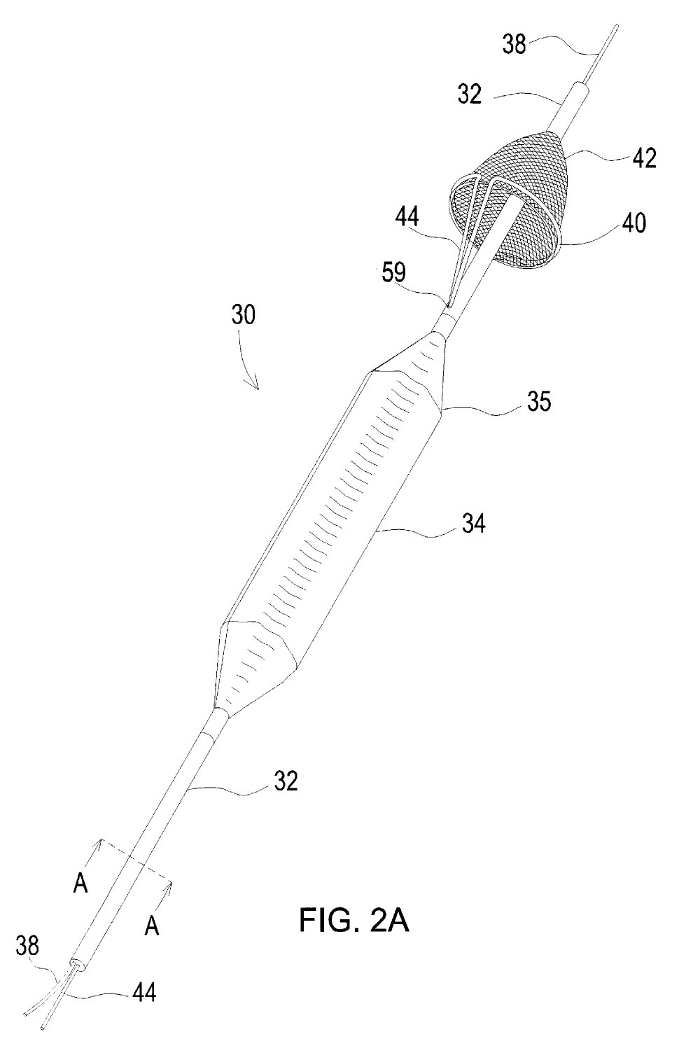

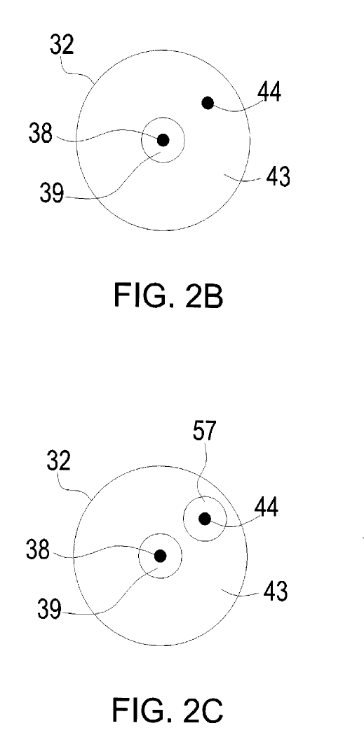

Referring now to FIG. 2A, there is shown a perspective view of a device 30 for balloon angioplasty with an embolization protection component according to a preferred embodiment of the present invention. The inventive device 30 comprises a shaft 32, dilatation balloon 34, retraction filament 44, flexible filter 42 and filter deployment ring 40. In FIG. 2A, device 30 is depicted mounted on a guide wire 38.

Filter 42 may optionally comprise either a shape memory metal, comprising an alloy such as copper-zinc-aluminium-nickel, copper-aluminium-nickel, or nickel-titanium, (for example, Nitinol), or a mesh of very thin polymeric material (for example, ultra high molecular weight polyethylene). The pores of the filter should be of sufficient size to allow free passage of blood cells, while trapping atherosclerotic debris and thrombi emboli. Preferably, the pores are in the range of 30 to 50 microns.

As seen in FIG. 2A, dilatation balloon 34 is mounted on shaft 32, which extends beyond the distal end 35 of balloon 34. The diameter of shaft 32 is selected to enable deployment of shaft 32 within an artery. For example, the diameter of a shaft for deployment within a coronary artery is approximately 1 millimeter, or less.

As known in the art of balloon angioplasty, shaft 32 includes a guide wire lumen 39 (shown in FIGS. 2B and 2C) from a proximal end of shaft 32 passing through the distal tip of shaft 32, allowing balloon 34 to be guided along a deployed guidewire to a location in the vasculature. Additionally, as known in the art of balloon angioplasty, shaft 32 also includes an inflation fluid lumen 43 (see FIGS. 2B and 2C) providing fluid communication with the inner volume of balloon 34, allowing inflation and deflation of balloon 34 by introduction or removal of inflation fluid from the inner volume of balloon 34.

Device 30 further comprises a retraction filament 44 allowing an operator to collapse and/or release filter deployment ring 40. Retraction filament 44 passes from the proximal end to the distal end of shaft 32 through a lumen in shaft 32, which is separate from guide wire lumen 39, and emerges from an opening 59 in shaft 32 to attach to filter deployment ring 40.

As shown in FIG. 2B, the lumen in shaft 32 through which retraction filament 44 passes may optionally comprise inflation fluid lumen 43, which is preferably coaxial with guide wire lumen 39, such that shaft 32 comprises at least two lumens.

Alternatively, as shown in FIG. 2C, retraction filament 44 may pass through a dedicated lumen 57 in shaft 32, isolated from inflation fluid lumen 43, such that shaft 32 comprises at least three lumens.

Filter deployment ring 40, which controls deployment and collapsing of filter 42, is disposed around the proximal opening of filter 42, and comprises a shape memory metal such as Nitinol or the like. When retraction filament 44 is pulled so as to tighten filter deployment ring 40, filter 42 is retained in a collapsed form.

When retraction filament 44 is released, filter deployment ring 40 returns to its original size and shape, thereby releasing pressure on filter 42, which is allowed to return to its original conical shape.

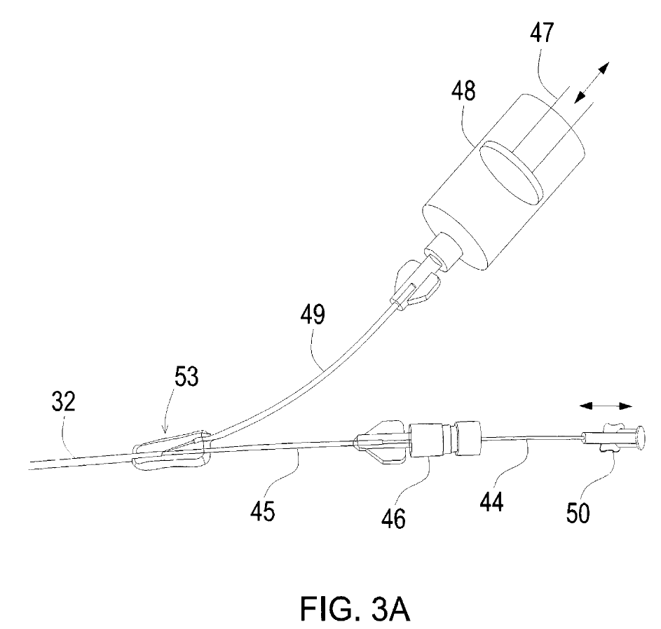

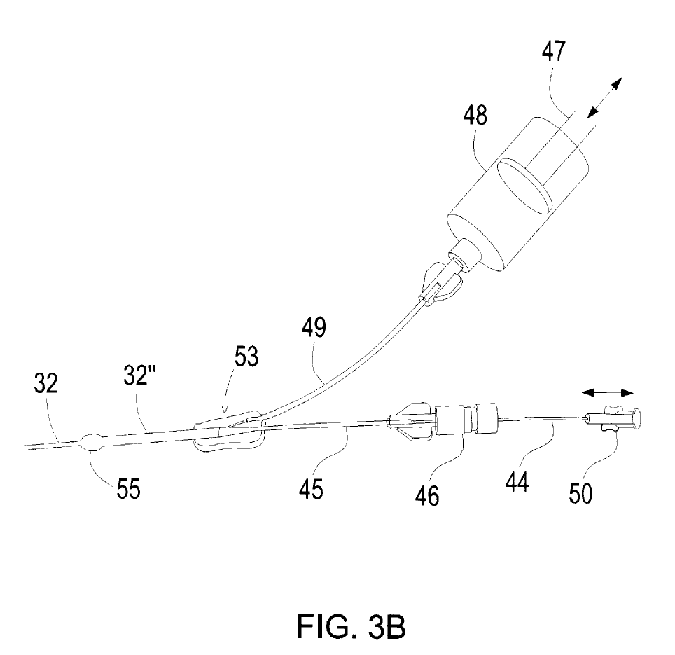

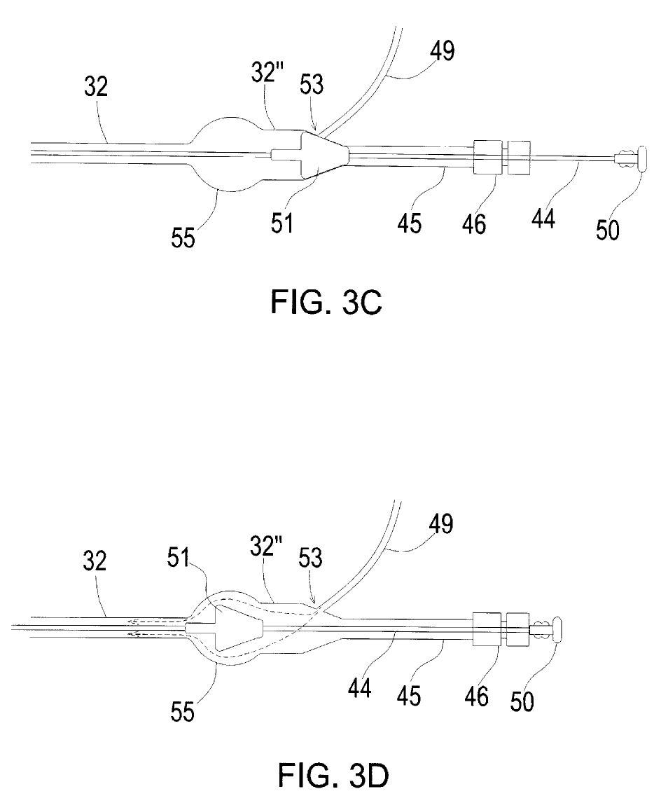

Referring now to FIG. 3A, there is shown the proximal part of the embolization device 30. The retraction filament 44 has a handle 50 on its proximal side, for moving the retraction filament 44 back and forth. The retraction filament 44 enters a filament tube 45 at the point where an external locking device 46 is situated. Locking device 46 can be twisted to the right or to the left, to lock and unlock the retraction filament 44 onto filament tube 45, so that the ability to move the retraction filament 44 back and forth is controlled. Retraction filament 44 covered by filament tube 45 enters shaft 32. Filament tube 45 may optionally be continuous with retraction filament lumen 57 of FIG. 2A or 2B.

As part of the device 30 there is provided a balloon inflation/deflation device 48 configured as a syringe having a plunger 47. The balloon inflation/deflation device 48 is attached to an inflation tube 49 entering shaft 32 at connection point 53. The inflation device is provided for the purpose of inflating balloon 34 by pushing fluid through tube 49 into balloon 34 via the hollow portion of shaft 32, and for deflating balloon 34 by withdrawing the fluid from balloon 34.

Referring now to FIGS. 3B-3C-3D, there is shown the device 30 as shown in FIG. 3A, modified to accommodate a feature of the invention which is the provision of a safety stopper 51, which is optionally substantially conical-shaped. The stopper 51 is responsible for ensuring that inflation of the balloon 34 will occur only once filter 42 is deployed, and not prior to deployment.

To accommodate stopper 51, section 32″ of shaft 32 is provided with a width slightly greater than that of shaft 32, beyond connection point 53. A portion of section 32″ is shaped to form a bulbous area 55, and distal to area 55 shaft 32 narrows and continues with the same width of the shaft 32 of FIG. 3A.

The following description discloses the mode of operation of safety stopper 51.

When retraction filament 44 is pulled, safety stopper 51, made of rubber, silicon or the like, is situated at connection point 53, thus blocking the entrance of the inflation fluid into section 32″ so that it is not possible to inflate balloon 34. At this point, filter 42 is closed, since retraction filament 44 is pulled. Once locking device 46 is unlocked and then retraction filament 44 is released and moved forward, it moves the stopper 51 along with it, since they are connected to each other. The stopper 51 moves into section 32″, thereby clearing the connection point 53 and allowing fluid to pass therethrough.

As stated, section 32″ is slightly wider beyond connection point 53, in order for the stopper 51 to fit therein. At the connection point 53, stopper 51 fits the internal circumference of section 32″ of shaft 32, so it blocks any fluid from entering it. After the wider section 32″ of shaft 32, there is a bulbous shaped area 55.

When filter 42 is being deployed, stopper 51 is pushed into area 55 with the forward movement of filament 44. Bulbous shaped area 55 is large enough to contain stopper 51 and also allow fluid to flow around the stopper 51, so that when stopper 51 is pushed to bulbous area 55, shaft 32 is not blocked and balloon 34 inflation is allowed. Distal to bulbous area 55, the shaft 32 is narrower than section 32″. The length of section 32″ plus bulbous area 55 is equal to the length that retraction filament 44 is pushed forward to fully deploy filter 42.

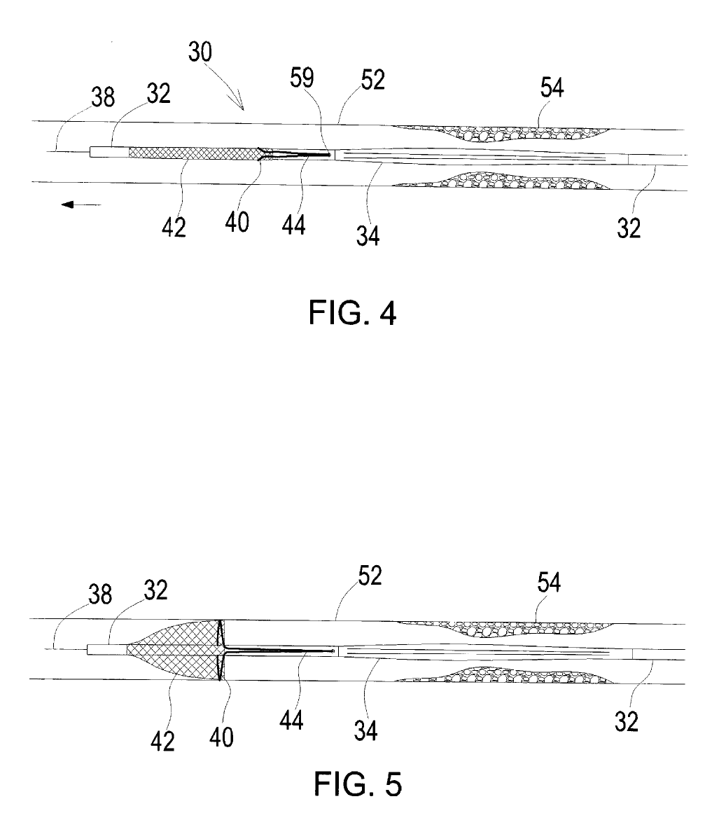

FIGS. 4-9 illustrate the use of device 30 in an angioplasty procedure.

Referring now to FIG. 4, there is shown the device 30 at the initial stage of an angioplasty procedure, with non-inflated balloon 34 lodged in a blood vessel 52, with the balloon 34 positioned where the atherosclerotic plaque 54 is obstructing blood vessel 52. Filter 42 is collapsed and retained by retraction filament 44.

The size in diameter of the non-inflated balloon 34 is 0.5-0.7 millimeters for coronary balloons and 1.1-1.5 millimeters for coronary stents. For intra-cerebral arteries the size of the device is similar. For larger arteries like carotid or peripheral arteries, the size may increase 2-3 fold.

Referring now to FIG. 5, there is shown the device 30 during an angioplasty procedure, when filter 42 has been deployed by unlocking the locking device 46 and releasing the retraction filament 44 through shaft 32. Once the filament 44 is released, the filter deployment ring 40 surrounding filter 42, is no longer tightly drawn by the retraction filament 44, and ring 40 is free to return by memory to its original shape, allowing filter 42 to deploy so as to fit the internal circumference of the blood vessel 52. Thus filter 42 is released from its collapsed form.

The size of filter 42 before deployment is 0.5-1 millimeter in diameter. The maximal size of the filter at the ring site when it is deployed is 2.5-5 millimeter in diameter for coronary arteries.

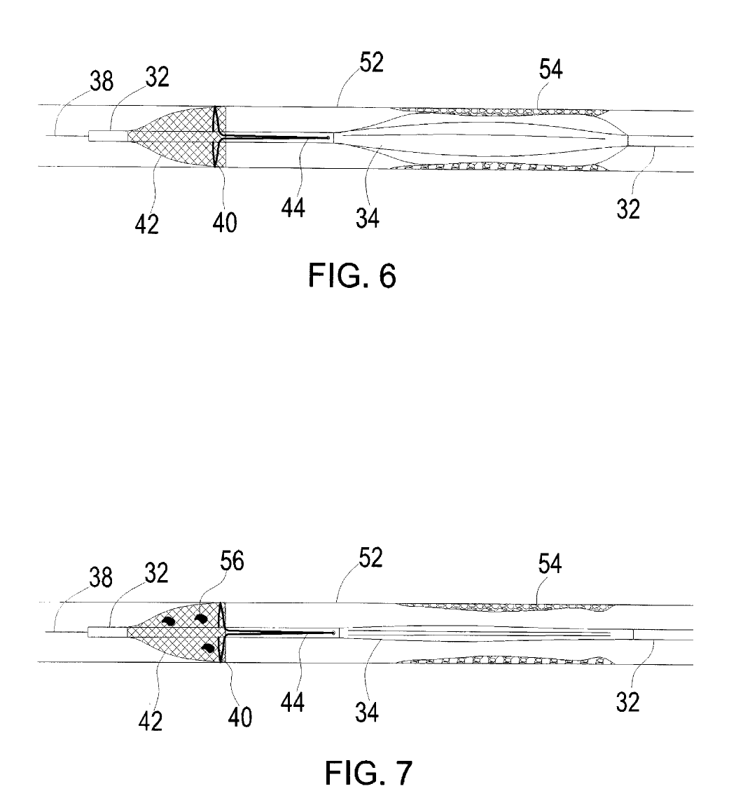

Referring now to FIG. 6, there is shown the device 30 with balloon 34 inflated and therefore pressing against plaque 54 on the walls of blood vessel 52. The pressure against the plaque 54 causes it to break up into small pieces which when they migrate with the blood flow are called emboli 56 (see FIG. 7). The size of the inflated balloon 34 is approximately 2-5 mm in diameter .

The sizes of both balloon 34 and filter 42 are 2-5 times larger when the angioplasty procedure is to be done in other arteries like carotid, renal or the peripheral arteries.

Referring now to FIG. 7, there is shown the device 30 with deflated balloon 34, and the layer of atherosclerotic plaque 54 is reduced because it was crushed and compressed by balloon 34, while inflated. The still deployed filter 42 traps any emboli 56 that are released from plaque 54, while allowing blood to continue flowing without interference through the porous filter. These emboli 56, if allowed to remain in blood vessel 52, can block smaller vessels downstream to blood vessel 52, and may cause, for example, infarction or stroke (depending on the treated artery) that may ultimately result in death.

In accordance with the principles of the present invention, the risk of small vessel downstream blockage by released emboli from the crushed plaque is eliminated since the filter deployment ring 40 seals the artery distally to the treatment site and the emboli flow into the porous filter wherein theyi are trapped. The present invention thus provides a device that traps the emboli and allows the blood to flow freely during the angioplasty procedure, thus eliminating the risk of downstream blockage and preserving flow during the procedure.

The balloon 34 inflation-deflation cycle can be repeated as many times as necessary while the filter is still deployed.

The size of the deflated balloon 34 (after initial inflation) is approximately 1-1.5 mm in diameter for coronary arteries, 2-5 times larger for other arteries.

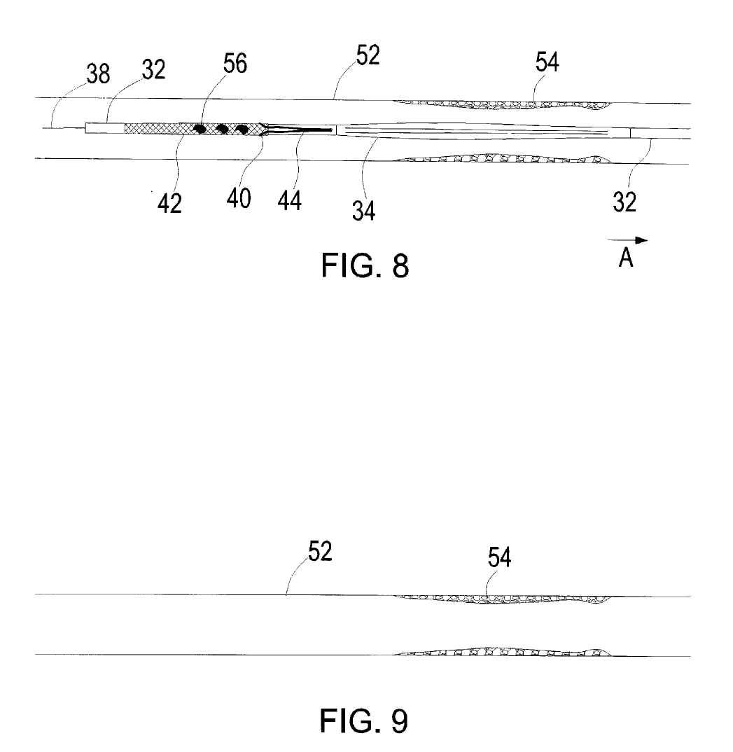

Referring now to FIG. 8, there is shown device 30 after completion of the angioplasty procedure. The balloon 34 has been deflated and filter 42 is collapsed, containing in it trapped emboli 56. The filter 42 is closed by pulling retraction filament 44, and therefore tightening the filter deployment ring 40, surrounding filter 42, causing filter 42 to collapse to a small profile of 1-1.5 mm in diameter.

The device 30 is shown being retracted from vessel 52 (arrow A).

Referring now to FIG. 9, there is shown blood vessel 52, after removal of the device 30. As a result of the treatment, the layer of atherosclerotic plaque 54 is reduced and therefore vessel 52 is less obstructed and blood can flow more freely through it.

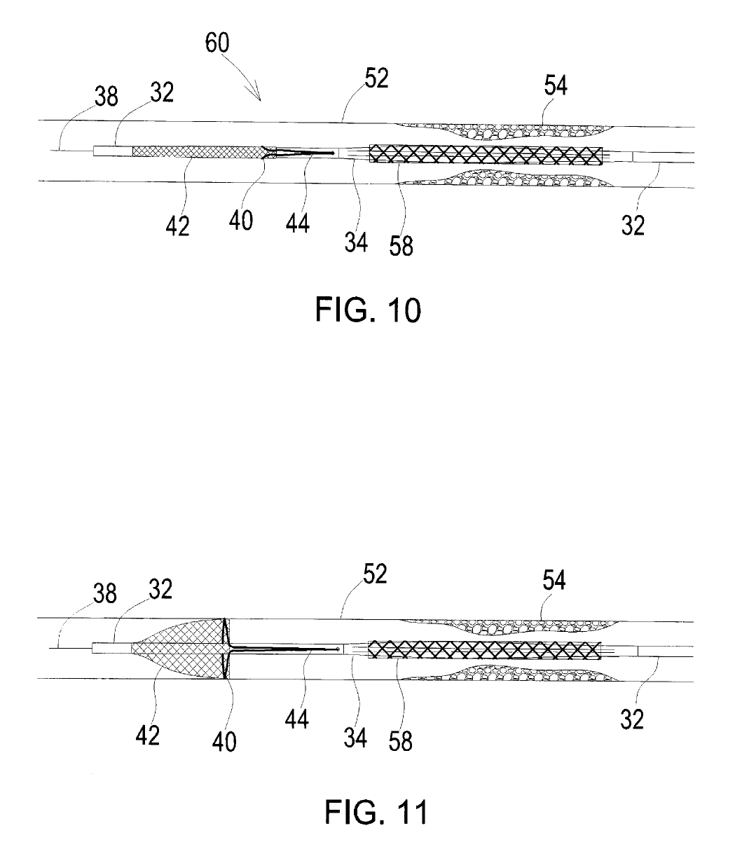

FIGS. 10-15 illustrate another preferred embodiment of the device 30 for use in an angioplasty procedure with the addition of a stent.

Referring now to FIG. 10, there is shown an alternative embodiment of the present invention, featuring a dilatation balloon and stent 58 with an embolization protection device 60. As shown, the device 60 is not yet deployed and is lodged in blood vessel 52 at the initial stage of the procedure.

Referring now to FIG. 11, there is shown the device 60 of FIG. 10, with filter 42 deployed, by releasing filter deployment ring 40 by the release of retraction filament 44.

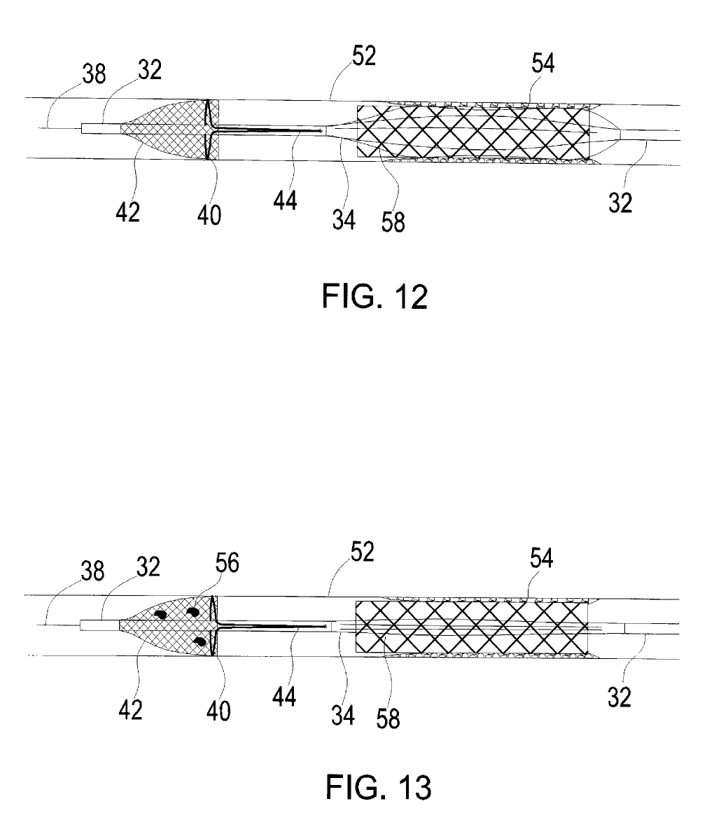

Referring now to FIG. 12, there is shown the device 60 with balloon 34 inflated, causing stent 58 mounted on balloon 34 to deploy and to expand to fit the circumference of constricted vessel 52 and crush against plaque 54 on the walls of blood vessel 52, and thereby open the plaque narrowing the artery. During this process the plaque 54 is broken into small pieces called emboli 56.

Referring now to FIG. 13, there is shown the device 60, with balloon 34 deflated, thus releasing emboli 56 from plaque 54 flowing downstream to balloon 34 and getting trapped by deployed filter 42. The porous filter 42 allows blood to flow through it and therefore does not block the blood flow.

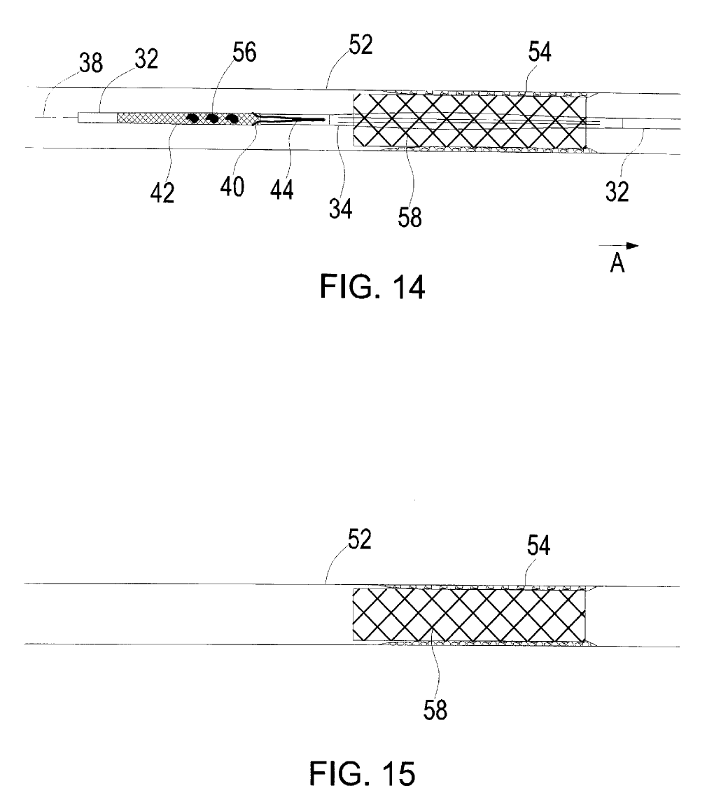

Referring now to FIG. 14, there is shown the device 60 with filter 42 closed, containing trapped emboli 56 in it. The filter 42 is closed by pulling retraction filament 44 which in turn pulls filter deployment ring 40 which is disposed around proximal opening of filter 42. The filter 42 collapses into a small profile, as in the first embodiment (FIG. 8). In this embodiment, the small profile of the closed filter 42 is essential so as to avoid getting entangled with stent 58 when it is withdrawn from vessel 52, through deployed stent 58.

The device 60 is retracted from blood vessel 52, as indicated by arrow A.

Referring now to FIG. 15, there is shown the stent 58 deployed at the plaque 54 site, after treatment, thus keeping the treatment site open.

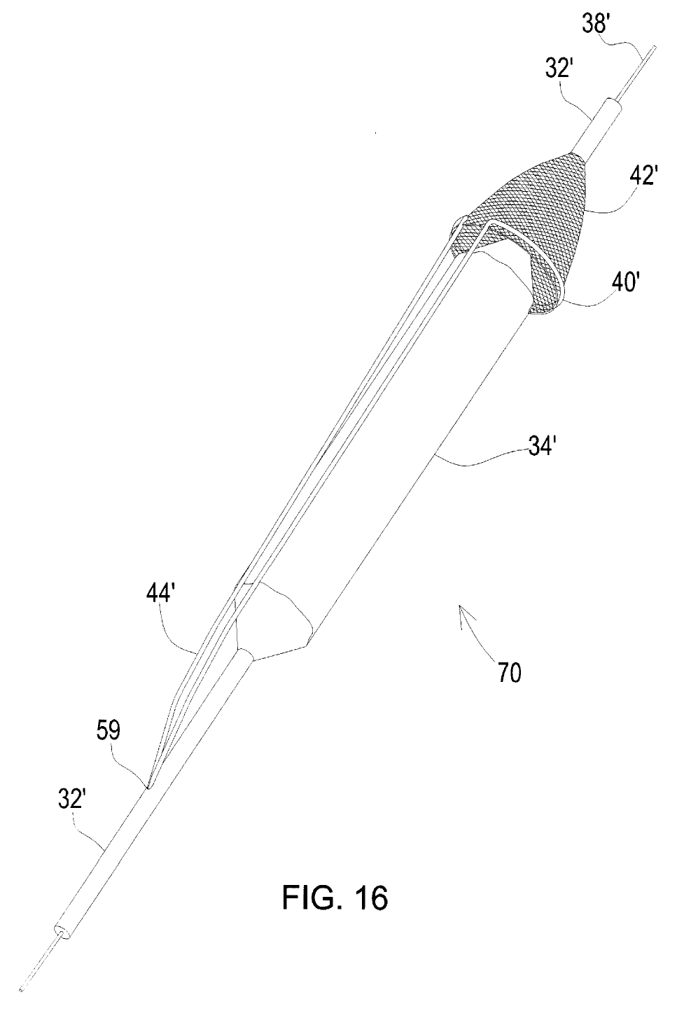

Referring now to FIG. 16, there is shown a perspective view of a device 70 for balloon angioplasty according to a preferred embodiment of the present invention. The device is meant only for a balloon without a stent, in an alternative to the embodiment of FIG. 2.

The retraction filament 44′ is released from an opening 59 in shaft 32′ proximal to balloon 34′, and is attached to a thin metal memory filament and continues all the way to filter 42′ which is distal to balloon 34′, where it forms a ring and surrounds it as described in FIG. 2. Filter 42′ surrounds a portion of the distal end of balloon 34′, so that when balloon 34′ is inflated it further deploys filter 42′ by pushing against it.

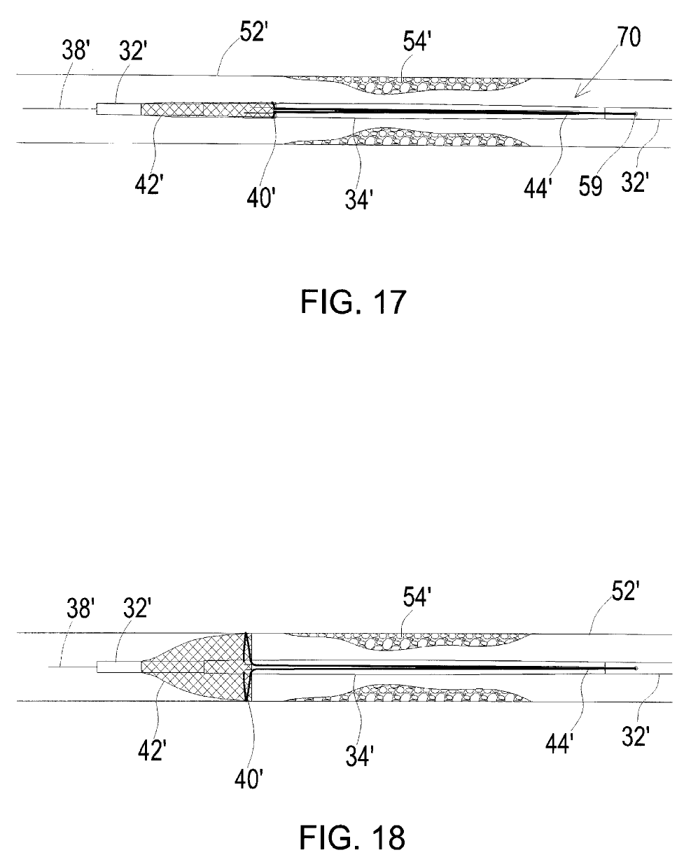

Referring now to FIG. 17, there is shown device 70 lodged in blood vessel 52′, in the initial stage of the angioplasty procedure, as described in FIG. 4.

Referring now to FIG. 18, there is shown device 70 with filter 42′ deployed, prior to balloon 34′ inflation.

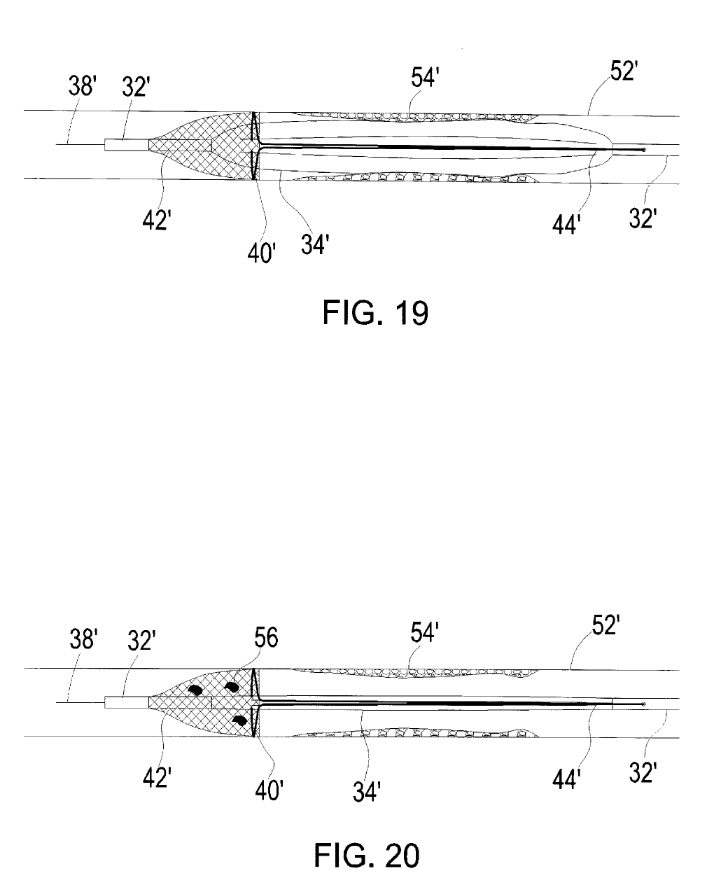

Referring now to FIG. 19, there is shown device 70 with balloon 34′ inflated. Since the balloon 34′ is partially surrounded by the filter 42′, the balloon 34′ comes into contact with filter 42′ as it is inflated, and this inflation further deploys filter 42′

Referring now to FIG. 20, there is shown balloon 34′ deflated, thereby releasing emboli particles 56 from the crushed plaque 54′ which are trapped in still-deployed filter 42′, with filter 42′ allowing blood to flow through it so as not to obstruct the blood flow.

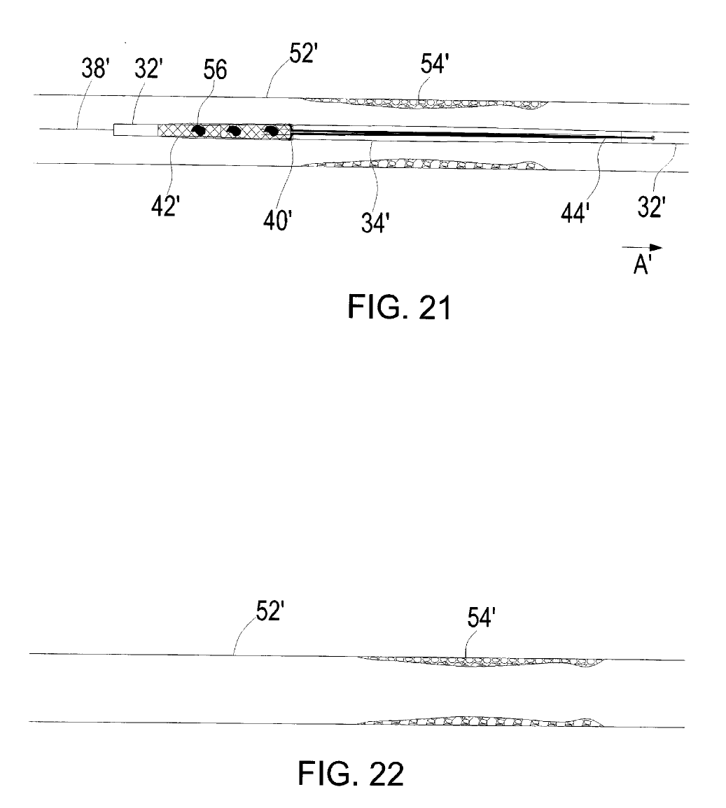

Referring now to FIG. 21, there is shown filter 42′ collapsed, with emboli 56 trapped within, being withdrawn from vessel 52′ (arrow A′).

Referring now to FIG. 22, there is shown blood vessel 52′ having dilated plaque 54′ after successful balloon angioplasty treatment.

The inventive distal embolization protection component, according to the preferred embodiments of the present invention, can be used for any stent over balloon deployment system known in the art, as well as any endoluminal method that involves balloon inflation inside any blood vessel like coronary, intra-cerebral arteries, carotid, renal and alike.

Although particular embodiments of the invention have been described and illustrated herein, it is recognized that modifications and variations may readily occur to those skilled in the art and consequently, it is intended that the claims be interpreted to cover such modifications and equivalents.

I claim:

1. An angioplasty device for providing protection against distal embolization of atherosclerotic debris and thrombi emboli resulting from an angioplasty procedure, said device comprising:
an endoluminal dilatation balloon mounted on a shaft, said shaft having at least one lumen;
a flexible filter mounted on said shaft, distal to said balloon; and
a filter deployment ring encircling a proximal end of said flexible filter for the purpose of controlling deployment and collapsing of said filter, said filter deployment ring comprising shape memory metal,
wherein said flexible filter, when deployed, traps said atherosclerotic debris and thrombi emboli thus allowing blood flow to continue without interference during said angioplasty procedure.

2. The device of claim 1, wherein said filter deployment ring is connected to a thin retraction filament extending through said at least one lumen in said shaft and emerging from an opening in said shaft distal to said balloon.

3. The device of claim 2, wherein said at least one lumen comprises an inflation fluid lumen.

4. The device of claim 2, wherein said at least one lumen comprises a dedicated lumen.

5. The device of claim 3, wherein said filter deployment ring is tightened by pulling said retraction filament connected to said filter deployment ring.

6. The device of claim 2, wherein said tightened filter deployment ring causes said filter to close, containing in it said trapped atherosclerotic debris and thrombi emboli.

7. The device of claim 2, further comprising a separate retraction filament tube proximal to said balloon, said tube joining said shaft, wherein said retraction filament extends through said shaft, and a locking device enabling and disabling forward and backward movement of said retraction filament, wherein said locking device locks said retraction filament onto said retraction filament tube into through which said retraction filament passes.

8. The device of claim 1, further comprising a balloon inflation/deflation device configured as a syringe having a plunger proximal to said shaft, and connected to said shaft via an inflation tube, wherein said balloon inflation/deflation device injects inflation fluid through said inflation tube into said balloon for inflating it, and withdrawing said fluid from said balloon for deflating it.

9. The device of claim 1, wherein said balloon is reversibly inflatable.

10. The device of claim 7, wherein said filter deployment ring is deployed by unlocking said locking device and thereby releasing said thin retraction filament connected to said filter deployment ring, and thus said ring returns to its original shape by memory.

11. The device of claim 8, further comprising a safety stopper for the purpose of ensuring that said deployment of said filter occurs before said balloon inflation, wherein said safety stopper is attached to said retraction filament and is situated at the connection point of said inflation tube with said shaft, thus blocking the entrance of said inflation fluid for inflating said balloon, when said retraction filament is pulled and said filter is not yet deployed.

12. The device of claim 11, wherein said safety stopper is pushed forward with the forward movement of said retraction filament when deploying said filter, and said stopper is then removed from said connection point of said inflation tube to said shaft so that said inflation fluid is allowed to flow through said shaft and inflate said balloon.

13. The device of claim 11, wherein said shaft is wider in diameter beyond said connection point for the purpose of accommodating said safety stopper.

14. The device of claim 13, further comprising a bulbous area formed in said shaft situated distally to said wider section of said shaft, wherein said bulbous area is large enough to contain said stopper once said filter is deployed and allow fluid to flow through it, and wherein distally to said bulbous area said shaft continues at a regular width.

15. The device of claim 11 wherein said safety stopper is made of at least one of silicon and rubber.

16. The device of claim 13 wherein the length of said wider section of said shaft together with the length of said bulbous area is equal to the distance said retraction filament is pushed forward when said filter is being deployed.

17. The device of claim 1, wherein said balloon distal end is partially surrounded by said filter so that when said balloon is inflated it will further deploy said filter by pushing against it.

18. The device of claim 1, further comprising a stent that is pre-crimped over said balloon, and deployment of said stent is achieved by inflation of said balloon.

19. The device of claim 1, wherein a diameter of said shaft is approximately one millimeter.

20. The device of claim 1, wherein a diameter of said balloon before inflation is 0.5 to 0.7 millimeters for coronary balloons, and a diameter of said balloon when inflated is approximately 2 to 4 millimeters, and a diameter said balloon after deflation is approximately 2.5 millimeters.

21. The device of claim 18, wherein the diameter of said balloon for coronary stents is 1.1 to 1.5 millimeters.

22. The device of claim 1, wherein said filter deployment ring comprises a shape memory metal and said flexible filter is made of one of a shape memory metal and a very thin polymeric material.

23. The device of claim 1, wherein said flexible filter before deployment is 0.5 to 1 millimeter in diameter, and the maximal size of said filter at the filter deployment ring site when it is deployed is 2.5 to 5 millimeters in diameter.

24. The device of claim 1, wherein the size of said filter before deployment is 0. 5 to 1 millimeter in diameter, and the maximal size of said filter at said memory ring site is 2.5 to 5 millimeters in diameter.

25. The device of claim 1, wherein said filter deployment ring comprises a shape memory metal.

26. A method for performing an angioplasty procedure and providing protection against distal embolization resulting in said angioplasty procedure, said method comprising:
inserting an endoluminal dilatation balloon mounted on a haft into a blood vessel obstructed by plaque, said shaft having a flexible filter mounted thereon, distal to said balloon;
deploying said flexible filter by releasing a filter deployment ring disposed around a proximal end of said flexible filter for the purpose of controlling deployment and collapsing of said filter; and
inflating said dilatation balloon so that it expands and crushes said plaque,
wherein said deployed flexible filter, traps atherosclerotic debris and thrombi emboli that are released from said crushed plaque when said balloon is deflated, thus allowing blood flow to continue without interference during said angioplasty procedure.

27. The method of claim 26, wherein said endoluminal dilatation balloon is withdrawn from said blood vessel after performance of said angioplasty procedure.

28. The method of claim 26, wherein said releasing of said filter deployment memory ring is achieved by unlocking a locking device which locks a thin retraction filament extending from an opening distal to said balloon in said shaft, onto a filament tube through which retraction filament passes, wherein said retraction filament is attached to said filter deployment memory ring, and thus by releasing said retraction filament said filter deployment memory ring is also released and allowed to expand by memory.

29. The method of claim 28, wherein said filter deployment memory ring is collapsed by pulling said retraction filament, thereby collapsing said filter.

30. The method of claim 26, wherein said endoluminal dilatation balloon has a pre-crimped stent thereon, and by inflating said balloon, said stent is deployed.

31. The method of claim 30, wherein said endoluminal dilatation balloon is withdrawn from said blood vessel after performance of said angiangioplasty procedure, and said deployed stent is remained in said blood vessel.
